(19) United States
(12) Patent Application Publication
(10) Pub. No.: US 2024/0310254 A1
(43) Pub. Date: Sep. 19, 2024

(54) SOIL SAMPLE PURIFICATION DEVICE

(71) Applicant: NORTHWEST INSTITUTE OF ECO-ENVIRONMENT AND RESOURCES, CAS, Lanzhou (CN)

(72) Inventors: Caixia ZHANG, Lanzhou (CN); Ningjing TAN, Lanzhou (CN)

(21) Appl. No.: 18/676,476

(22) Filed: May 28, 2024

(30) Foreign Application Priority Data
Jun. 9, 2023 (CN) 202310681924.1

(51) Int. Cl. G01N 1/34; G01N 33/24

(52) U.S. Cl. CPC G01N 1/34; G01N 33/24

(57) ABSTRACT

A soil sample purification device includes a liquid supplying mechanism and a liquid feeding mechanism. The liquid supplying mechanism includes fluid injection channels, a fluid collector, a mounting plate and fluid supplying boxes. The fluid supplying boxes are movably connected to the mounting plate, and are configured to respectively carry a single fluid material. The fluid collector is provided with a fluid collecting chamber, which is communicated with the fluid supplying boxes respectively via the fluid injection channels. The fluid supplying boxes are configured to move between a fully-loaded position and an unloaded position. The liquid feeding mechanism includes a liquid feeding box and a plugging member. The plugging member is movably provided on the liquid feeding box, and can move in a specified direction to block the outlet channel, or move in a direction opposite to the specified direction to unblock the outlet channel.

### CROSS-REFERENCE TO RELATED APPLICATIONS

This application claims the benefit of priority from Chinese Patent Application No. 202310681924.1, filed on Jun. 9, 2023. The content of the aforementioned application, including any intervening amendments thereto, is incorporated herein by reference in its entirety.

### TECHNICAL FIELD

This application relates to purification devices, and more specifically to a soil sample purification device.

### BACKGROUND

For the particle size measurement, the soil sample is required to be free of residual organic matters, and the calcium concretions are required to be removed. In addition, it is also required to disperse the soil aggregate s to allow more accurate determination of the particle size distribution characteristics of the soil.

In the actual experiments, the above requirements are generally satisfied by temperature-control heating with an electric heating plate and the introduction of hydrogen peroxide, hydrochloric acid, and dispersant. However, since reactions in the soil are slow, the whole process needs to be artificially monitored, and pure water needs to be added constantly during the heating operation of the electric heating plate to prevent the beaker from being dry. The whole process is time-consuming and laborious, and the use of the electric heating plate brings potential safety problems.

### SUMMARY

An objective of the present disclosure is to provide a soil sample purification device to overcome the deficiencies in the prior art.

Technical solutions of the present disclosure are described below.

This application provides a soil sample purification device, comprising:
a liquid supplying mechanism; and
a liquid feeding mechanism;
wherein the liquid supplying mechanism comprises a plurality of fluid injection channels, a fluid collector, a mounting plate and a plurality of fluid supplying boxes; the plurality of fluid supplying boxes are movably connected to the mounting plate; each of the plurality of fluid supplying boxes is configured to carry a fluid material; the fluid collector is provided with a fluid collecting chamber; the plurality of fluid supplying boxes are communicated with the fluid collecting chamber respectively via the plurality of fluid injection channels; each of the plurality of fluid supplying boxes is configured to move between a fully-loaded position and an unloaded position; and a volume of the fluid material loaded by the plurality of fluid supplying boxes at the fully-loaded position is larger than a volume of the fluid material loaded by the plurality of fluid supplying boxes at the unloaded position; and
the liquid feeding mechanism comprises a liquid feeding box and a plugging member; the liquid feeding box is provided with a liquid feeding chamber and an outlet channel communicated with the liquid feeding chamber; the plugging member is movably provided on the liquid feeding box, and is capable of moving in a first designated direction to block the outlet channel, or moving in a direction opposite to the first designated direction to unblock the outlet channel; the fluid collector is detachably connected to the liquid feeding box; and the fluid collector is configured to supply the fluid material to the liquid feeding box.

In an embodiment, the liquid supplying mechanism further comprises a sensing mechanism and an indicating mechanism; the sensing mechanism is configured to detect the volume of the fluid material in the plurality of fluid supplying boxes; the indicating mechanism is connected to the sensing mechanism; the indicating mechanism is configured to send indicating information based on a signal sent by the sensing mechanism; and the indicating information is configured to indicate the volume of the fluid material in the plurality of fluid supplying boxes.

In an embodiment, a plugging structure is provided on a side of the outlet channel communicated with the liquid feeding chamber; the plugging member is provided with a plugging end; and an inner contour of the plugging structure is configured to fit an outer contour of the plugging end.

In an embodiment, the liquid feeding mechanism further comprises a driving mechanism; the driving mechanism is in transmission connection with the plugging member; and the driving mechanism is configured to drive the plugging member to move in the first direction.

In an embodiment, the soil sample purification device further comprises a vessel.

In an embodiment, the soil sample purification device further comprises a heating mechanism, and the heating mechanism is configured to heat the vessel and the fluid material in the vessel.

In an embodiment, the soil sample purification device further comprises a shaking mechanism, and the shaking mechanism is configured to shake the fluid material in the vessel.

In an embodiment, the soil sample purification device further comprises a fixing mechanism. The vessel is configured to be arranged in the fixing mechanism in a specified direction and/or a designated sequence; and the fixing mechanism is configured to restrict the vessel.

In an embodiment, the soil sample purification device further comprises a waste liquid collection mechanism, and the waste liquid collection mechanism is at least configured to carry a waste liquid.

In an embodiment, the waste liquid collection mechanism is provided with a waste liquid storage chamber and a plurality of flushing mechanisms; and the plurality of flushing mechanisms are disposed opposite to each other.

In an embodiment, the fixing mechanism comprises a fixing plate and a sensing mechanism; the fixing plate is provided with a plurality of accommodating holes arranged in a specified direction and/or at a specified spacing; the plurality of accommodating holes are configured to accommodate the vessel; and the sensing mechanism is configured to detect a level of the fluid material in the vessel.

In an embodiment, the sensing mechanism comprises an infrared sensing mechanism; the infrared sensing mechanism comprises a transmitting end and a receiving end; the receiving end is configured to receive infrared rays emitted by the transmitting end; and at least a portion of the vessel is placed on a transmission path of the infrared rays.

In an embodiment, the soil sample purification device further comprises a transferring mechanism; the transferring mechanism comprises an articulated arm or at least two gripping arms capable of moving relative to each other; the articulated arm or the at least two gripping arms are capable of moving in an X-axis and/or the Y-axis and/or the Z-axis direction driven by a driving mechanism; and the articulated arm or the at least two gripping arms are detachably fixed to the vessel and/or to the fixing mechanism and/or to the liquid feeding mechanism.

In an embodiment, the soil sample purification device further comprises a positioning structure; the positioning structure comprises a first fixing portion and a second fixing portion; the first fixing portion is able to be clamped to the second fixing portion; the first fixing portion is provided on the articulated arm or the at least two gripping arms; and the second fixing portion is provided on the liquid feeding box and/or the fixing mechanism.

Compared with the prior art, the present disclosure has at least the following advantages.

The soil sample purification device provided herein is provided with a liquid supplying mechanism and a liquid feeding mechanism, such that multiple samples respectively in multiple vessels can be treated simultaneously, allowing for high efficiency and simple operation.

### BRIEF DESCRIPTION OF THE DRAWINGS

FIG. 1 is a schematic diagram of a soil sample purification device according to an embodiment of the present disclosure;

FIG. 2 is a schematic diagram of a liquid supplying mechanism according to an embodiment of the present disclosure;

FIG. 3 is a schematic diagram of a liquid feeding mechanism according to an embodiment of the present disclosure;

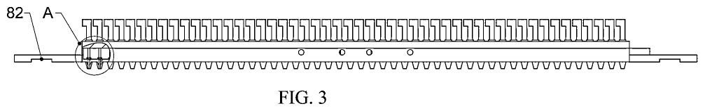

FIG. 4 is an enlarged view of portion A in FIG. 3;

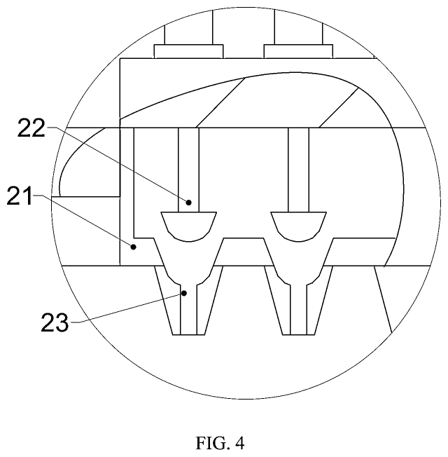

FIG. 5 is a partially enlarged view of the liquid feeding mechanism according to another embodiment of the present disclosure;

FIG. 6 is a schematic diagram of a fixing mechanism according to an embodiment of the present disclosure;

FIG. 7 is a partially enlarged view of the fixing mechanism according to an embodiment of the present disclosure;

FIG. 8 is a schematic diagram of a waste liquid collection mechanism according to an embodiment of the present disclosure; and

FIG. 9 is a schematic diagram of a transferring mechanism according to an embodiment of the present disclosure.

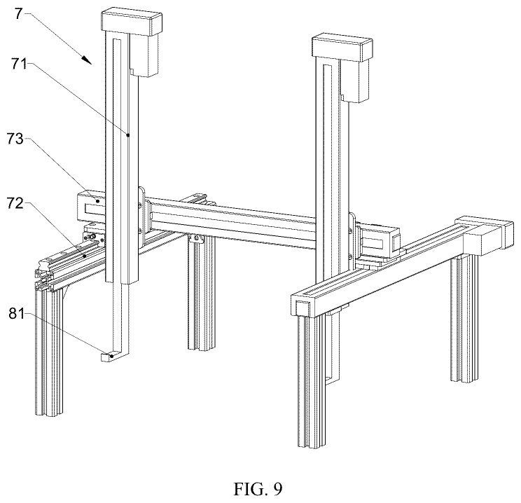

In the drawings:
1, liquid supplying mechanism; 11, fluid collector; 12, mounting plate; 13, fluid supplying box; 14, liquid collecting tube; 16, ventilating channel;
2, liquid feeding mechanism; 21, liquid feeding box; 22, plugging member; 23, outlet channel;
3, heating mechanism;
4, shaking mechanism;
5, fixing mechanism; 51, fixing plate; 52, transmitting end; 53, receiving end;
6, waste liquid collection mechanism; 61, waste liquid storage chamber; 62, flushing mechanism;
7, transferring mechanism; 71, gripping arm; 72, first mounting bracket; 73, second mounting bracket; 74, third mounting bracket;
81, first fixing portion; 82, second fixing portion; and
9, vessel.

### DETAILED DESCRIPTION OF EMBODIMENTS

In view of deficiencies in the prior art, technical solutions of the present disclosure are proposed herein. The technical solutions, and implementations and principles thereof will be further explained below.

### Embodiment 1

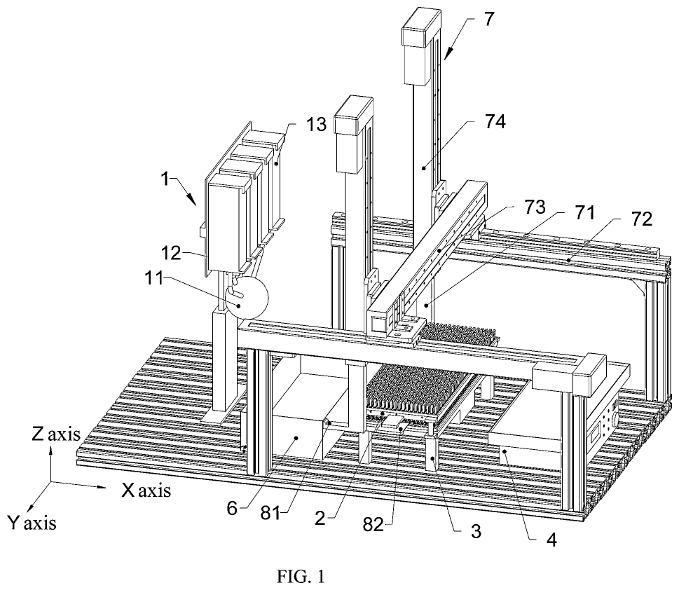

Referring to an embodiment shown in FIG. 1, a soil sample purification device is provided, which includes an operating platform, a liquid supplying mechanism 1, a liquid feeding mechanism 2, a heating mechanism 3, a shaking mechanism 4, a fixing mechanism 5, a waste liquid collection mechanism 6, and a transferring mechanism 7.

Understandably, at least one of the liquid supplying mechanism 1, the waste liquid collection mechanism 6, the heating mechanism 3, the shaking mechanism 4, and the transferring mechanism 7 is detachably connected to the operating platform. More specifically, the operating platform may be provided with a slideway along an X-axis and a Y-axis, and the liquid supplying mechanism 1, the waste liquid collection mechanism 6, the heating mechanism 3, the shaking mechanism 4, and the transferring mechanism 7 may be moved and fixed to a specified position on the operating platform through the slideway. FIG. 1 shows the slideway provided along the X-axis. The position relationships among the liquid supplying mechanism 1, the waste liquid collection mechanism 6, the heating mechanism 3, the shaking mechanism 4 and the transferring mechanism 7 can be adjusted according to the actual experimental and operational requirements.

Specifically, a to-be-treated soil sample can be placed in a vessel 9. The liquid supplying mechanism 1 is configured to provide the same kind of fluid material or different kinds of fluid materials for the liquid feeding mechanism 2. The liquid feeding mechanism 2 can be communicated with the liquid supplying mechanism 1 to add the same kind of fluid material or different kinds of fluid materials to a designated vessel 9. One vessel 9 or a plurality of vessels 9 can be operated in an experiment, and the plurality of vessels 9 can be fixedly arranged on the fixing mechanism 5 at a designated position or in a sequence. The transferring mechanism 7 can be fixed with the vessel 9, the fixing mechanism 5 and the liquid feeding mechanism 2 to realize the movement of the fixing mechanism 5 and the liquid feeding mechanism 2. When the vessel 9 needs to be heated, the transferring mechanism 7 can be used to transfer the vessel 9 to the heating mechanism 3, or the transferring mechanism 7 can be fixed with the fixing mechanism 5 and can transfer the plurality of vessels 9 constrained by the fixing mechanism 5 to the heating mechanism 3 by transferring the fixing mechanism 5. The heating mechanism 3 can heat the vessel 9 and the fluid materials therein. When the vessel 9 needs to be shaken uniformly, the transferring mechanism 7 can either transfer the vessel 9 directly or transfer the fixing mechanism 5, to which the plurality of vessels 9 are fixed, to the shaking mechanism 4 such that the fluid materials in the vessel 9 are shaken uniformly through the shaking mechanism 4. After the heating operation and the shaking operation of the purification experiment are completed, the supernatant in the vessel 9 can be sucked out by the liquid supplying mechanism 1, and then can be discharged into the waste liquid collection mechanism 6. The soil sample purification device provided herein can simultaneously operate a plurality of vessels 9 in the same group of experiments. Compared with manual addition of materials, the device provided herein can quickly and efficiently complete addition of materials to a plurality of vessels 9 and suck out the supernatant by setting the liquid supplying mechanism 1, the liquid feeding mechanism 2 and the waste liquid collection mechanism 6.

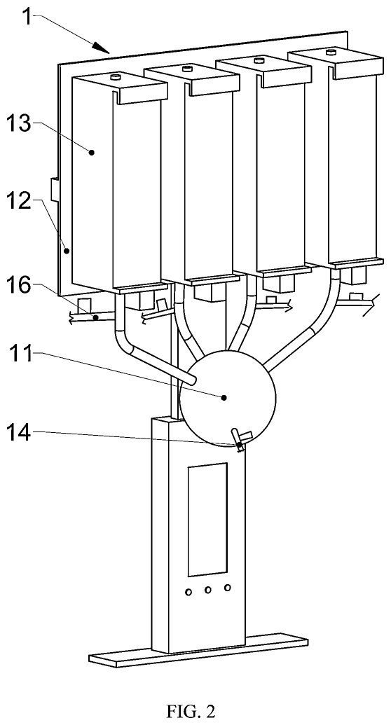

More specifically, as shown in FIG. 2, the liquid supplying mechanism 1 includes a mounting plate 12, a fluid collector 11, a plurality of fluid supplying boxes 13 and a connecting frame. The mounting plate 12 is slidably provided on the connecting frame and is capable of sliding in a specified direction. As shown in FIG. 2, the specified sliding direction may be a Z-axis direction, and a driving device may be provided for transmission connection with the mounting plate 12. The driving device is configured to drive the mounting plate 12 to move along the Z-axis direction to adjust the position of the plurality of fluid supplying boxes 13. The connecting frame may also include a plurality of sliding rails (not shown in the drawings) along the X-axis, Y-axis and Z-axis, and the mounting plate 12 can be driven by the driving device to slide along the X-axis, Y-axis and Z-axis, so as to adjust the position of the fluid collector 11 and the plurality of fluid supplying boxes 13, which facilitates the addition of the fluid material, and avoids the interfering and colliding of the components.

Preferably, the fluid collector 11 may be a hollow sphere.

The plurality of the fluid supplying boxes 13 are slidably provided on the mounting plate 12, and the fluid collector 11 can be communicated with the plurality of fluid supplying boxes 13 through fluid injection channels. Each of the plurality of fluid supplying boxes 13 can provide a single fluid material for the fluid collector 11. In the actual experiments, a plurality of fluid supplying boxes 13 can be pre-provided, and each of the plurality of fluid supplying boxes 13 can be provided with the required fluid material. When the fluid material in one of the plurality of fluid supplying boxes 13 is exhausted, the fluid supplying box can be directly added with the fluid material or can be replaced with another fluid supplying box for quick replenishment. In a preferred embodiment, each of the plurality of fluid supplying boxes 13 is also connected to a resilient member, and the resilient member can be a spring, a torsion spring, or an air pump. In an embodiment, the resilient member is a spring. When one fluid supplying box 13 is loaded with a larger amount of fluid material, the fluid supplying box 13 is in a fully-loaded position, and the fluid supplying box 13 and the fluid material therein compress the spring. With the gradual outflow of the fluid material in the fluid supplying box 13, the pressure received by the spring decreases, such that the fluid supplying box 13 is able to move from the fully-loaded position to the unloaded position under the driving force of the spring. In this case, the staff can judge the usage of the fluid material by checking the position of the fluid supplying box 13.

More specifically, the liquid supplying mechanism 1 further includes a first sensing mechanism and an indicating mechanism. It is understood that the first sensing mechanism is used for detecting the capacity of the fluid material in the fluid supplying box 13 and feeding back the detected signal to the indicating mechanism. The fluid supplying box 13 can be filled with a specified capacity of the fluid material. When the first sensing mechanism detects that the fluid material in the fluid supplying box 13 is exhausted or relatively less, it will trigger the indicating mechanism to send a corresponding indication information based on the signal sent by the first sensing mechanism, and the indication information may indicate the capacity of the fluid material in the fluid supplying box 13. Specifically, the first sensing mechanism may be a liquid level sensor, and the liquid level sensor may be provided in the fluid supplying box 13. The indication information may be sound, light or image. When the fluid material in the fluid supplying box 13 is less, the liquid level sensor detects that the liquid level in the fluid supplying box 13 is lowered to a to-be-indicated position, triggering the indicating mechanism to indicate the situation through alarming, light blinking, constant lighting or graphic display to indicate to the staff that the fluid material in the fluid supplying box 13 is about to run out. The first sensing mechanism may also be a gravity sensor, and the gravity sensor may be provided on the moving path of the fluid supplying box 13. When the fluid supplying box 13 moves from the fully-loaded position to the unloaded position due to the discharge of the fluid material, the gravity sensor detects that the fluid material in the fluid supplying box 13 is about to run out, and thus triggers the indicating mechanism to indicate the alarm.

More specifically, the fluid collector 11 may also be provided with a liquid collecting tube 14 and a switching valve. The liquid collecting tube 14 is provided extending out of the fluid collector 11. One end of the liquid collecting tube 14 is connected to the fluid collector 11, and the other end of the liquid collecting tube 14 may be detachably connected to the liquid feeding box 21 of the liquid feeding mechanism 2, so that the fluid collector 11 may be communicated with the liquid feeding box 21 when in use, and may be connect the liquid feeding box 21 with the fluid collector 14 when not in use, thereby facilitating the connection between the fluid collector 11 and the liquid feeding box 21. The switching valve is provided on the liquid collecting tube 14, and the flow rate and the on/off of the liquid collecting tube 14 can be controlled by the on/off of the switching valve.

More specifically, as shown in FIG. 2, the liquid supplying mechanism 1 further includes a ventilating channel 16 and a switching valve. The ventilating channel 16 is communicated with the fluid injection channel. The switching valve can be provided between the ventilating channel 16 and the fluid injection channel, and can be switched between a first position and a second position. When the switching valve is in the first position, it can be understood that when the switching valve is switched on, the ventilating channel 16 will connect the fluid injection channel and the external atmospheric environment. The external atmospheric pressure can accelerate the discharge of fluid materials in the fluid collecting chamber. When the switching valve is in the second position, it can be understood that when the switching valve is closed, the fluid injection channel is closed, that is, the supply of fluid materials from the fluid supplying box 13 to the fluid collector 11 is stopped.

More specifically, referring to FIG. 2, an embodiment provided herein provides four fluid supplying boxes 13, each of which accommodates a single fluid material. The fluid materials in the four fluid supplying boxes 13 are different. Of course, the fluid supplying box 13 can also be one or more, and the number and arrangement of which can be configured according to the actual experimental and operational requirements. The plurality of fluid supplying boxes 13 can contain the same or different kinds of fluid materials. Each fluid supplying box 13 is communicated with the fluid collector 11 via a liquid supplying pipe, and the liquid supplying pipe is provided with a liquid feeding flow channel. The fluid supplying boxes 13 can feed the specified fluid material into the fluid collector 11 via specified liquid supplying pipes. Specifically, when adding fluid materials, the fluid supplying box 13 can provide a quantitative and corresponding type of fluid materials to the fluid collector 11 through manual intervention or programmable logic controller (PLC) control. In some operations requiring fluid materials for reaction, the fluid collector 11 can also be used as a reactor, and a quantitative and specified type of fluid materials are fed to the fluid collector 11 in batches to carry out the reaction.

In a preferred embodiment, the liquid feeding mechanism 2 includes a liquid feeding box 21 and a plugging member 22. The liquid feeding box 21 is provided with a liquid feeding chamber, a discharging nozzle, and an outlet channel 23. The discharging nozzle is in communication with the liquid feeding chamber. The discharging nozzle is provided with the outlet channel 23, and the outlet channel 23 is in communication with the liquid feeding chamber. The discharge nozzle protrudes from an outer surface of the liquid feeding box 21 and is at least configured to extend into the vessel 9. The plugging member 22 is movably connected to the liquid feeding chamber, and the outlet channel 23 of the liquid feeding chamber corresponds to a designated vessel 9. The plugging member 22 can seal the outlet channel 23 and close the liquid feeding chamber. When the fluid collecting chamber is in communication with the liquid feeding chamber, the fluid material in the fluid collector 11 can be channeled into the liquid feeding chamber. When the plugging member 22 leaves the position of blocking the outlet channel 23, the outlet channel 23 is in communication with the liquid feeding chamber, and the fluid material in the liquid feeding chamber can flow from the outlet channel 23 into the vessel 9. By switching different fluid supply boxes 13 to provide different kinds of fluid materials to the fluid collector 11, different kinds of fluid materials enter into the vessel 9 in batches, so as to feed different fluid materials in batches as required for the experiment, which is more time-saving and labor-saving compared to manual material addition.

More specifically, the outlet channel 23 is provided with a plugging structure near a side of the liquid feeding chamber. The plugging member 22 can be plugged with the plugging structure through the plugging end of the plugging member 22. The outer contour of the plugging end is adapted to the inner contour of the plugging structure. When the plugging end moves toward the plugging structure, the distance between the plugging end and the plugging structure decreases, so that the fluid material can flow between the plugging end and the plugging structure and ultimately flows out of the outlet channel 23. The plugging member may be in transmission connection with a driving mechanism. Specifically, the driving mechanism can be a servo motor, and the servo motor is in transmission connection with the plugging member 22. The servo motor can drive the plugging member 22 to move and plug the outlet channel 23 or leave the position of blocking the outlet channel 23, so as to control the flow rate of the liquid discharged from the outlet channel 23.

More specifically, the radial size of the plugging end is reduced in the direction of gravity.

In a preferred embodiment, a plurality of vessels 9 are required for the experiment, and the plurality of vessels 9 can be arranged sequentially in a specified direction to form an area to be fed with liquid having a corresponding length in the X-axis and the Y-axis. The liquid feeding box 21 is provided with a plurality of outlet channels 23, and a feeding port of each outlet channel 23 corresponds to a vessel 9. More specifically, the soil sample purification device further includes a first pneumatic mechanism and a second pneumatic mechanism. The first pneumatic mechanism and the second pneumatic mechanism may be an air pump, so that when the supernatant in the vessel 9 is required to be sucked out, the fluid collector 11 is connected to the liquid feeding box, and the liquid feeding chamber is connected to the first pneumatic mechanism. Gas is passed to the liquid feeding box 21 through the first pneumatic mechanism, so that the fluid material in the liquid feeding chamber can enter the fluid collecting chamber more quickly. The second pneumatic mechanism is in communication with the fluid injection channel or the fluid collector 11, which can pump gas outwardly from the fluid collecting chamber, and accelerate the entry of the fluid material in the liquid feeding chamber into the fluid collecting chamber.

More specifically, the fixing mechanism 5 of the soil sample purification device includes a fixing plate 51 provided with a plurality of accommodating holes, and a second sensing mechanism. The plurality of accommodating holes can be provided according to the layout requirement for the experiment or commonly used arrangement for the experiment. The arrangement includes a specified direction, a specified order, a specified specification, and a specified spacing. The second sensing mechanism includes an infrared sensing mechanism. The infrared sensing mechanism includes a transmitting end 52 and a receiving end 53. The receiving end 53 is configured to receive infrared rays emitted by the transmitting end 52. The vessel 9 may be placed between the receiving end 53 and the transmitting end 52, such that the accommodating space of the vessel 9 is placed in the transmission path of the infrared rays. The vessel 9 may be a beaker. Normally, when the beaker is empty, the infrared rays emitted by the transmitting end 52 will be transmitted to the receiving end 53 under the refraction of the two layers of glass, but when the liquid level floods the infrared ray path, the medium through which the infrared rays pass is changed to change the optical path, and the receiving end 53 will not be able to receive the infrared signals emitted by the transmitting end 52. At this time, the second sensing mechanism may send out an alarm through a reminder controller to prompt the operator that the required amount of liquid is reached. The second sensing mechanism can also be connected with a PLC controller. The PLC controller is connected with the liquid supplying mechanism 1 and the liquid feeding mechanism 2. When the second sensing mechanism senses that the amount of liquid has reached the required level, it can allow the liquid supplying mechanism 1 to stop supplying the liquid, and the liquid feeding mechanism 2 to stop refilling the liquid.

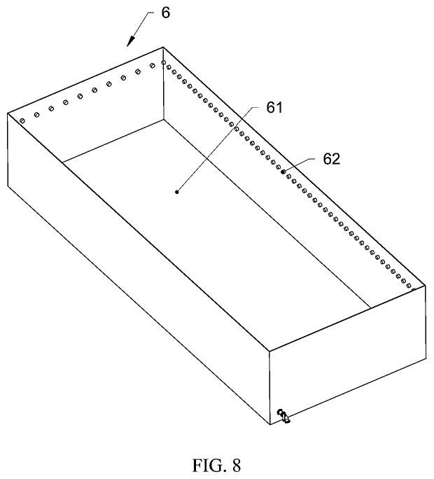

In a preferred embodiment, referring to FIG. 8, the waste liquid collection mechanism 6 of the soil sample purification device further includes a waste liquid collection box. The waste liquid collection box is provided with a waste liquid storage chamber 61, and waste liquid in the fluid collecting chamber can be discharged into the waste liquid storage chamber 61. More specifically, the waste liquid collection mechanism 6 also includes a plurality of flushing mechanisms 62. The plurality of flushing mechanisms 62 can be evenly arranged along the circumferential direction of the waste liquid collection box, and can spray oriented towards the center of the waste liquid collection box. The liquid collecting tube 14 of the fluid collector 11 can be placed in the spray path of the flushing mechanisms 62, so as to facilitate the flushing of the liquid collecting tube 14 of the fluid collector 11 to avoid interference of waste liquids with the subsequent experiments. During the experiment, the liquid collecting tube 14 can pump and discharge pure water repeatedly, so that the pure water enters the fluid collector 11 to flush the inner wall of the fluid collector, so as to achieve a better cleaning effect.

Specifically, the waste liquid collection box includes a high-pressure flushing region and a waste liquid collection region sequentially arranged in the direction of gravity. The high-pressure flushing region is connected to the flushing mechanism 62 to provide high-pressure water to the flushing mechanism 62. The waste liquid collection region forms the waste liquid storage chamber 61 for containing waste liquid. The waste liquid collection region may also be connected to a drain device, and the drain device may be a faucet. Waste liquid in the waste liquid collection region may be discharged through the drain device.

In a preferred embodiment, the transferring mechanism 7 can be a robot. The transferring mechanism 7 provided in this embodiment includes a gripping arm 71, a first mounting bracket 72, a second mounting bracket 73, and a third mounting bracket 74. The first mounting bracket 72 is provided with a first sliding rail provided in the X-axis direction. The second mounting bracket 73 is movably arranged on a first sliding rail and is capable of moving in the X-axis direction. The second mounting bracket 73 is provided with a second sliding rail provided in the Y-axis direction. The third mounting bracket 74 is movably arranged on the second sliding rail and is capable of moving in the Y-axis direction. The third mounting bracket 74 is provided with a third sliding rail provided in the Z-axis direction. The gripping arm 71 is movably provided on the third sliding rail and is capable of moving in the Z-axis direction. Understandably, the gripping arm 71 can move along the X-axis, the Y-axis and the Z-axis, and the gripping arm 71 can move along the X-axis, the Y-axis and the Z-axis. Y-axis and Z-axis to transfer the vessel 9, the fixing mechanism 5 and the liquid feeding mechanism 2.

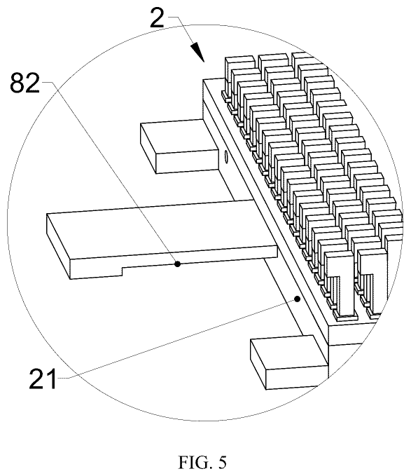

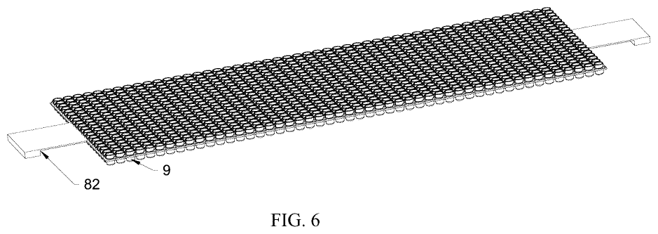

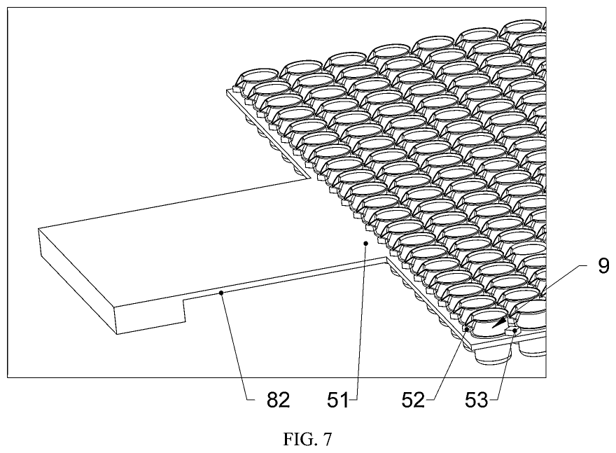

More specifically, referring to FIGS. 5-7, the gripping arm 71 is fixed to the liquid feeding box 21 or the fixing mechanism 5 through fitting of a positioning structure, so that the liquid feeding box 21 or the fixing mechanism 5 can be stably fixed to the gripping arm 71. The positioning structure includes a first fixing portion 81 and a second fixing portion 82. The first fixing portion 81 is provided on the gripping arm 71, and the second fixing portion 82 is provided on the liquid feeding box 21 and fixing mechanism 5. The first fixing portion 81 may be provided with a recess, and the second fixing portion 82 may be provided with a tab that fits with the contour of the recess.

More specifically, as shown in FIG. 5, the second fixing portion 82 includes at least two balancing portions and a fitting portion. The fitting portion and the balancing portion are provided in sequence along the direction of gravity. The fitting portion is provided with a sliding through groove, and the two balancing portions are provided on two sides of the fitting portion respectively, and may be provided symmetrically on two sides of the fitting portion. For example, the second fixing portion 82 provided on the liquid feeding mechanism 2, the balancing portion and the fitting portion are all provided on the liquid feeding box 21, and are provided extending in the same direction. The sliding through groove extends outwardly from the liquid feeding box 21, where the extending direction is defined as a length direction of the sliding through groove. The balancing portion extends outwardly from the liquid feeding box 21, where the extending direction is defined as a length direction of the balancing portion. The length of the sliding through groove is larger than that of the balancing portion. The first fixing portion 81 is provided with a snapping through groove, and the snapping through groove is able to snap with the sliding through groove. When the fitting portion is snapped with the first fixing portion 81, the gripping arm 71 can move towards the side close to the liquid feeding box 21 to enable the gripping arm 71 to be constrained between the fitting portion and the balancing portion, so that the liquid feeding box 21 can be more stably fixed between the two gripping arms 71.

In practice, four fluid materials are placed in four fluid supplying boxes 13, respectively. The fluid supplying boxes 13 are in communication with the fluid collector 11, and the fluid collector 11 is also in communication with the liquid feeding boxes 21. The plugging member 22 plugs the outlet channels 23 on the liquid feeding boxes 21, and the liquid feeding boxes 21 are provided with a plurality of outlet channels 23. Each outlet channel corresponds to a vessel 9, so that the fluid supplying boxes 13 supply a specified amount of fluid material to the liquid feeding boxes 21 through the fluid collector 11. The specified amount of fluid material may be greater than the sum of all fluids actually injected into the vessel 9, i.e., the amount of fluid material in the liquid feeding box 21 may be greater than actually required amount of fluid material. The rate of fluid material discharged by the fluid supplying box 13 into the vessel 9 is controlled by controlling the distance between the plugging member 22 and the outlet channel 23. After the fluid injection is completed, the outlet channels 23 are blocked by the plugging member 22. After that, two fluid materials injected successively react with each other, or to avoid mutual contamination or interference of the two different fluid materials, cleaning water can be supplied to the fluid supplying box 13. After repeated flushing, the cleaned waste liquid can be discharged into the waste liquid collection box.

The fixing mechanism 5 is accommodated with a plurality of vessels 9. After the plurality of vessels 9 are rested, as shown in FIG. 1, the heating mechanism 3 and the shaking mechanism 4 are placed adjacent to each other. Before heating, the fixing mechanism 5 can be placed on the heating mechanism 3, but the heating mechanism is not turned on. When heating is required, the heating mechanism is turned on to perform the heating step under the specified heating conditions.

After the heating is finished, the above steps are repeated to add fluid material to the vessel 9. When the vessel is rested, the fluid material in the vessel 9 is layered to form an upper layer of clear liquid and a lower layer of mixture material. The liquid feeding box 21 is capable of moving in the vertical direction towards the fixing mechanism 5 under the clamping of the transferring mechanism 7, while the discharge nozzle extends into the vessel 9 to suck up the upper layer of clear liquid in the vessel 9 to complete the sucking step of the upper layer of clear liquid.

The transferring mechanism 7 is configured to clamp the fixing mechanism 5 and place it on the shaking mechanism 4 for shaking to complete the shaking step.

### Example 2

In this embodiment, the soil sample purification device provided by the present disclosure is applied to an experimental operation, and the specific operating steps are as follows.

Four fluid supplying boxes 13 are provided and contain 1000 ml of 10% hydrogen peroxide, hydrochloric acid, sodium hexametaphosphate, and ultrapure water respectively.

100 beakers with a capacity of 50 ml are provided on the fixing mechanism 5, and the openings of the beakers correspond to the outlet channels 23 of the liquid feeding boxed 21.

1000 ml of hydrogen peroxide is provided to the fluid collector 11 through the fluid supplying box 13, so that the hydrogen peroxide in the fluid collector 11 enters into the liquid feeding cavity, and 10 ml of hydrogen peroxide is added to each beaker. The transferring mechanism 7 is used to transfer the fixing mechanism 5 to the heating mechanism 3 for heating at 120° C. for 6-8 h. The above operations are repeated. 10 ml of hydrochloric acid is added to each beaker through the feeding mode of the fluid supplying box 13—the fluid collector 11—the liquid feeding box 21, and heated at 120° C. for 3 h. After that, each beaker is added with 40 ml of ultrapure water, followed by resting for 10-15 h. The upper layer of clear liquid is extracted from each beaker using the liquid feeding mechanism 2, so that the remaining liquid in the vessel 9 is 20 ml, and 10 ml of sodium hexametaphosphate solution is added and dispersed by ultrasonic vibration through the shaking mechanism 4 for 10 min, that is, the entire soil purification and dispersion process is completed. Of course, only hydrogen peroxide can be added to remove organic matters according to the actual sample conditions, or only hydrochloric acid can be added to remove calcium nodules. All these can be chosen flexibly according to the actual sample conditions.

Specifically, the heating mechanism 3 can be an electric heating plate with temperature control and timing functions, which can self-adjust the temperature and the heating time of each heating session. During the heating, ultrapure water can also be provided to the liquid feeding mechanism 2 through the liquid supplying mechanism 1, and can be added to the beaker through the liquid feeding mechanism 2, so as to prevent the sample in the beaker from being dry-burned.

The soil sample purification device provided herein can feed fluid material to a plurality of beakers simultaneously. Compared with the manual addition of fluid material, it is more efficient, and the plurality of beakers can carry out the subsequent operation process at the same time, which is more convenient for use.

The above embodiments are only to illustrate the technical concepts and features of the present disclosure, and are not intended to limit the scope of the present disclosure. It should be understood that any changes or modifications made in accordance with the spirit of the present disclosure shall be covered by the scope of the present disclosure defined by the appended claims.

What is claimed is:

1. A soil sample purification device, comprising:
a liquid supplying mechanism;
a liquid feeding mechanism;
a vessel;
a heating mechanism;
a shaking mechanism;
a fixing mechanism;
a waste liquid collection mechanism; and
a transferring mechanism;
wherein the liquid supplying mechanism comprises a plurality of fluid injection channels, a fluid collector, a mounting plate and a plurality of fluid supplying boxes; the plurality of fluid supplying boxes are movably connected to the mounting plate; each of the plurality of fluid supplying boxes is configured to carry a fluid material; the fluid collector is provided with a fluid collecting chamber; the plurality of fluid supplying boxes are communicated with the fluid collecting chamber respectively via the plurality of fluid injection channels; each of the plurality of fluid supplying boxes is configured to move between a fully-loaded position and an unloaded position; and a volume of the fluid material loaded by the plurality of fluid supplying boxes at the fully-loaded position is larger than a volume of the fluid material loaded by the plurality of fluid supplying boxes at the unloaded position;
the liquid feeding mechanism comprises a liquid feeding box and a plugging member; the liquid feeding box is provided with a liquid feeding chamber and an outlet channel communicated with the liquid feeding chamber; the plugging member is movably provided on the liquid feeding box, and is capable of moving in a first designated direction to block the outlet channel, or moving in a direction opposite to the first designated direction to unblock the outlet channel; the fluid collector is detachably connected to the liquid feeding box; and the fluid collector is configured to supply the fluid material to the liquid feeding box;
the heating mechanism is configured to heat the vessel and the fluid material in the vessel;
the shaking mechanism is configured to shake the fluid material in the vessel;
the vessel is configured to be arranged in the fixing mechanism in a second designated direction and/or a designated sequence; and the fixing mechanism is configured to restrict the vessel;
the waste liquid collection mechanism is at least configured to carry a waste liquid;
the transferring mechanism comprises an articulated arm or at least two gripping arms capable of moving relative to each other; the articulated arm or the at least two gripping arms are configured to be driven by a first driving mechanism to move in an X-axis direction, a Y-axis direction, a Z-axis direction or a combination thereof; and the articulated arm or the at least two gripping arms are detachably fixed to the vessel, the fixing mechanism, the liquid feeding mechanism or a combination thereof.

2. The soil sample purification device of claim 1, wherein the fluid supplying mechanism further comprises a sensing mechanism and an indicating mechanism; the sensing mechanism is configured to detect the volume of the fluid material in the plurality of fluid supplying boxes; the indicating mechanism is connected to the sensing mechanism; the indicating mechanism is configured to send indicating information based on a signal sent by the sensing mechanism; and the indicating information is configured to indicate the volume of the fluid material in the plurality of fluid supplying boxes.

3. The soil sample purification device of claim 1, wherein a plugging structure is provided on a side of the outlet channel communicated with the liquid feeding chamber; the plugging member is provided with a plugging end; and an inner contour of the plugging structure is configured to fit an outer contour of the plugging end.

4. The soil sample purification device of claim 1, wherein the liquid feeding mechanism further comprises a second driving mechanism; the second driving mechanism is in transmission connection with the plugging member; and the second driving mechanism is configured to drive the plugging member to move in the first designated direction.

5. The soil sample purification device of claim 1, wherein the waste liquid collection mechanism is provided with a waste liquid storage chamber and a plurality of flushing mechanisms; the plurality of flushing mechanisms are arranged in two rows; and those of the plurality of flushing mechanisms in one row are opposite to those of the plurality of flushing mechanisms in the other row, respectively.

6. The soil sample purification device of claim 1, wherein the fixing mechanism comprises a fixing plate and a sensing mechanism; the fixing plate is provided with a plurality of accommodating holes arranged in a third designated direction and/or at a designated spacing; the plurality of accommodating holes are configured to accommodate the vessel; and the sensing mechanism is configured to detect a level of the fluid material in the vessel.

7. The soil sample purification device of claim 6, wherein the sensing mechanism comprises an infrared sensing mechanism; the infrared sensing mechanism comprises a transmitting end and a receiving end; the receiving end is configured to receive infrared rays emitted by the transmitting end; and the vessel is configured to be at least partially located on a transmission path of the infrared rays.

8. The soil sample purification device of claim 7, further comprising:
a positioning structure;
wherein the positioning structure comprises a first fixing portion and a second fixing portion; the first fixing portion is configured to be clamped to the second fixing portion; the first fixing portion is provided on the articulated arm or the at least two gripping arms; and the second fixing portion is provided on the liquid feeding box and/or the fixing mechanism.
